(19) United States
(12) Patent Application Publication
(10) Pub. No.: US 2015/0133767 A1
(43) Pub. Date: May 14, 2015

(54) Biopsy Needle System for MR-Guided Biopsy

(71) Applicant: Agron Lumiani, Bielefeld (DE)

(72) Inventor: Agron Lumiani, Bielefeld (DE)

(21) Appl. No.: 14/536,785

(22) Filed: Nov. 10, 2014

(30) Foreign Application Priority Data
Nov. 13, 2013 (DE) 10 2013 112 471.2

(51) Int. Cl. G01R 33/28; A61B 10/02

(52) U.S. Cl. CPC G01R 33/287; A61B 10/0266
USPC 600/411

(57) ABSTRACT

The invention discloses a biopsy needle system (100, 200, 300), having: a hollow needle (100) having a cylindrical portion (102) adapted to be introduced into a patient, wherein the cylindrical portion comprises a first material; a first mandrin (200) comprising a sharp tip (204) and that may be introduced into the hollow needle (100) such that the tip (204) and an opposing end (206) of the first mandrin (200) project from the opposing end of the hollow needle (100), if the hollow needle (100) is located in the patient, wherein the sharp tip (204) is adapted to pierce tissue; characterized by an imaging mandrin (300) comprising a second needle and that may be inserted into the hollow needle (100) if the hollow needle (100) if the hollow needle is located in a patient, wherein the second material is a material that may be imaged appropriately during MR-imaging.

### CROSS-REFERENCE TO RELATED APPLICATION(S)

This application claims the benefit of German Patent Application No. DE 10 2013 112 471.2, filed Nov. 13, 2013, the entirety of which is hereby incorporated herein by reference.

### BACKGROUND OF THE INVENTION

1. Field of the Invention

The present invention relates to a biopsy needle for the MR-guided biopsy for application in a magnet resonance tomograph of arbitrary magnetic field strength, particularly having a magnetic field strength of 3 Tesla and more.

2. Description of the Related Art

During MR-guided biopsy a biopsy needle is moved by a physician in the direction of the lesion. In predetermined intervals it is verified by imaging methods using a magnet resonance tomograph that the biopsy needle is moved to the lesion. If necessary a physician can apply corrections to ensure that the needle is moved to the lesion.

In a magnet resonance tomograph signals of hydrogen atoms are generated under the influence of magnetic fields and dynamic magnetic fields, respectively in interaction with RF-radiation. The operation of a magnet resonance tomograph is known to the person skilled in the art and does not have to be explained in more detail herein.

Early magnet resonance tomographs had a magnetic field of 0.2 Tesla. Successively magnet resonance tomographs having a higher magnetic field strength became commercially available. Magnet resonance tomographs having a magnetic field strength of 1.0 Tesla and 1.5 Tesla have been introduced later and were accepted by the market and are worldwide available.

Magnet resonance tomographs having a magnetic field strength of 1.0 Tesla and 1.5 Tesla are increasingly replaced by magnet resonance tomographs having a field strength of 3 Tesla. Magnet resonance tomographs having a magnet field strength of 7 Tesla are also available.

Needles currently approved for MR-guided biopsy are made of titanium or comprise titanium coated portions. Such needles are MRT compatible up to a magnetic field strength of 1.5 Tesla. However, artifacts are caused in the region of the needles by these needles and a magnetic field strength of 1.5 Tesla.

A biopsy needle of the prior art for the MR-guided prostate biopsy comprises a hollow needle also termed coaxial portion. The biopsy needle further comprises a so-called mandrin having a sharp tip, which is introduced in use into the hollow needle and is used finally for punction. Both the hollow needle and the mandrin are as mentioned before made the by titanium or are titanium coated. Titanium is a non-ferromagnetic material.

During MR-guided biopsy the patient is lying on the table of the magnet resonance tomograph. At the patient so-called local coils may be arranged, for example. Before punction the patient is moved into the magnet resonance tomograph and imaging is performed by means of RF signals and gradients. After imaging the patient is moved out of the magnet resonance tomograph, wherein the patient keeps on lying on the table of the magnet resonance tomograph. The portion to be punctured is anesthetized locally and the skin is cut (incision) such that the hollow needle can enter the patient.

The physician introduces the hollow needle and the mandrin into the patient, wherein the patient is located outside of the magnet resonance tomograph.

When the physician has introduced the hollow needle and the mandrin by a predetermined distance into the patient, the physician can carry out a further imaging. Hereby, the patient is moved into the magnet resonance tomograph and in the region of its uniform magnet field, whereafter imaging is carried out by RF-signals and gradients signals.

Subsequently the physician can introduce the hollow needle and the mandrin further into the patient. The sequence of imaging and introducing the hollow needle including the mandrin may be repeated until the hollow needle has arrived at the lesion and the portion to be punctured, respectively. As soon as the hollow needle is positioned at the region to be punctured, which may be verified by imaging, the mandrin is removed and the hollow needle is fixed, for example by adhesive plaster. Subsequently, a biopsy needle is introduced into the hollow needle for removing tissue.

The MR-guided biopsy is known to the person skilled in the art and is not explained in further detail for the sake of brevity.

Magnet resonance tomograph having a magnetic field strength of 1.5 Tesla use radiofrequency signals having 63.87 MHz. Magnet resonance tomographs having a magnetic field strength of 3 Tesla use a radiofrequency signal having 127.73 MHz corresponding to a wave length of approximately 2.3 m. Such radiofrequency signal can heat a hollow needle and/or a mandrin, particularly if it comprises a length of approximately 16 cm to approximately 20 cm or more, which is inconvenient for the patient and may course in the worst case a local burn of the patient.

It is an object of the present invention to provide an improved biopsy needle system for MR-guided biopsy.

The object of the invention is achieved by a biopsy needle system according to claim 1, a hollow needle according to claim 8 and an imaging mandrin according to claim 10. The independent claims describe preferred embodiments.

### SUMMARY OF THE INVENTION

In one aspect of the invention, a cylindrical portion adapted to be introduced into a patient of an inventive hollow needle of a biopsy needle system is formed by a non-ferromagnetic and/or non-metallic material. Thereby, it may be ensured that the hollow needle is not heated by the high-frequency signal. Therefore, during MR-guided biopsy no unpleasant side effects for the patient occur. Burns may be avoided. Further, artifacts may be avoided.

The cylindrical portion of the hollow needle can be made of plastics, carbon, carbon fiber and/or ceramics. Particularly suited are hard plastics.

The object of the invention is also solved by an imaging mandrin adapted to be introduced into the above described hollow needle, wherein the mandrin comprises material that can be appropriately imaged by MR-imaging. The cylindrical portion, which is adapted to be introduced into a patient and which is made out of plastics, carbon, carbon fiber and/or ceramics, of the hollow needle can generally not be imaged by a magnet resonance tomograph.

The invention suggests providing beside the first mandrin having a sharp tip used for introducing the hollow needle and for biopsy, a further mandrin as imaging mandrin comprising a material that may be appropriately imaged using MR-imaging. The hollow needle can be introduced into the body of the patient, if the first mandrin having the sharp tip is projecting from the hollow needle, for example. For verifying the position of the hollow needle in the body of the patient the physician may remove the first mandrin having the sharp tip from the hollow needle and introduce the imaging mandrin having the material that may be imaged appropriately before imaging. Thereafter, imaging may be carried out by the magnet resonance tomograph. Based on the results of the imaging the physician can verify, whether the hollow needle is located at the appropriate location. If the hollow needle is located at the appropriate location, it can be assured that only tissue of the lesion and no healthy tissue is removed.

The imaging mandrin may comprise plastics. A cylindrical portion of the imaging mandrin may comprise plastics, wherein the cylindrical portion is adapted to be introduced into the hollow needle.

The imaging mandrin and the cylindrical portion of the imaging mandrin respectively may comprise a hollow portion in which a liquid and or a gel are located as a second exemplary material. The liquid and/or the gel may comprise oil, fish oil, water and/or contrast agent. A gel is a dispersion that may comprise a liquid. The gel may be medical gel, such as ultra noise gel. The gel may be hydrogel or lipogel. The hydrogel may comprise water. The lipogel may comprise plant oil, synthetic oil or mineral oil. The gel may further comprise methyldibromoglutaronitril, phenoxyethanol, benzyl alcohol and/or parbenes.

The invention also relates to a biopsy needle system comprising a hollow needle, a first mandrin having a sharp tip and an imaging mandrin. The first mandrin comprising the sharp tip may be introduced into the hollow needle such that the tip and an opposing end of the mandrin extend from opposing ends of the hollow needle, when the hollow needle is located in a patient or is introduced into the patient, wherein the sharp tip at the distal and is adapted to pierce, perforate and/or puncture tissue. The imaging mandrin comprises a second material and may be introduced into the hollow needle, if the hollow needle is located in a patient. The second material is a material that may be imaged appropriately to the using MR-10 imaging.

By imaging using a T1-weighted sequence a contrast agent comprising gadolinium may be imaged a appropriately. By a T1-weighted sequence also oil and fish oil may be appropriately imaged by a somewhat lower intensity. By imaging using a T2-15 weighted sequence oil, fish oil and water can be appropriately imaged. By a T2-weighted sequence also contrast agent comprising gadolinium can be imaged appropriately by a slightly lower intensity.

Using MR-imaging oil and fish oil can be imaged by a series of a T2-weighted sequence, a T1-weighted sequence and T1-FS weighted sequence, wherein FS is the appreciation for “Fat Suppression” and the order of the sequence is arbitrary. The contrast agent may be imaged by a series of a T1-weighted sequence, a T1-FS weighted sequence and a T2-weighted sequence, wherein the order of the sequence is also arbitrary.

In the context of the present invention appropriately imaged by MR-imaging means that the number and/or the density of the hydrogen atoms and/or of the protons in the second material is at least 5%, preferably at least 10%, preferably at least 15%, more preferred at least 25%, more preferred at least 30%, most preferred at least 50% higher or lower as the nuclear spin signal of the tissue around the hollow needle due to the hydrogen atoms and/or the protons and/or as the nuclear spin signal of the body tissue due to the hydrogen atoms and/or the protons therein. The body tissues may comprise soft tissue, organ tissue, skin tissue and/or nerve tissue.

In the context of the present invention appropriately imaged by MR-imaging means that the nuclear spin signal due to gadolinium in the second material is at least 5%, preferably at least 10%, more preferred at least 15%, more preferred at least 25%, more preferred at least 30%, most preferred at least 50% higher as the nuclear spin signal of the tissue around the hydrogen atoms and/or the protons around the hollow needle and/or as the nuclear spin signal of the body tissue due to the hydrogen atoms and/or the protons in the body tissue.

The hollow needle comprises a cylindrical portion adapted to be introduced into a patient. The cylindrical portion comprises a first material which is a non-ferromagnetic and/or a non-metallic material, such as plastics, carbon, carbon tissue and/or ceramics. The imaging mandrin comprises plastics. A cylindrical portion of the imaging mandrin may comprise plastics, wherein the cylindrical portion is adapted to be introduced into the hollow needle.

The imaging mandrin and the cylindrical portion respectively may comprise a cavity in which a liquid and/or a gel as a second exemplary material may be located. The liquid may comprise oil, fish oil, water and/or contrast agent, for example. The imaging mandrin comprises no sharp tip at the distal end and introduced into the patient and hollow needle. The distal end of imaging mandrin may be blunt.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a top view on the inventive hollow needle.

FIG. 2 is a top view of a mandrin.

FIG. 3 shows an imaging mandrin.

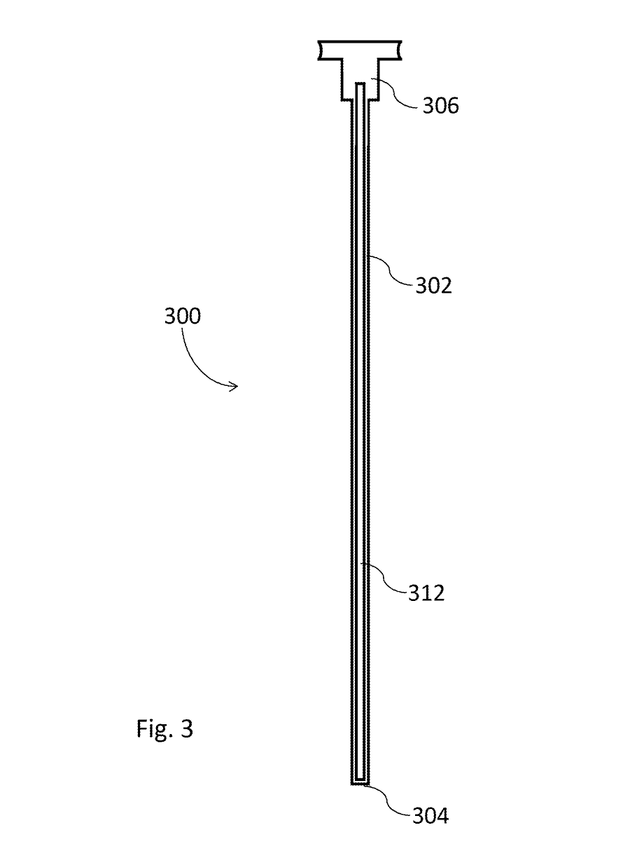

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

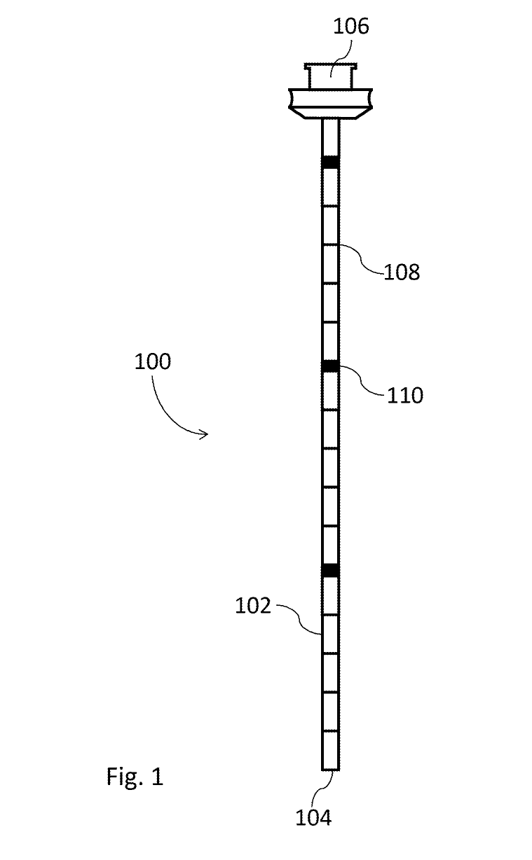

FIG. 1 shows a top view of a hollow needle 100 according to the present invention also termed cannula. The hollow needle 100 comprises a cylindrical portion 102, whose interior is hollow, a distal opening 104 to be introduced into the patient and a proximal grip portion 106 protruding in use from the patient. The cross-section of the cylindrical portion 102 is circular and the diameter of the cylindrical portion ranges in the field of prostate biopsy from approximately 1.5 mm to approximately 2 mm, for example. The length of the hollow needle 100 can range in the field of prostate biopsy from approximately 16 cm to approximately 20 cm or more, for example. It is to be understood that a hollow needle for biopsy of other organs may have different dimensions. The hollow needle may be manufactured from a non ferromagnetic and/or a non-metallic material. Particularly the cylindrical portion 102 can be made of a non ferromagnetic material and/or a non-metallic material. Thereby, it can be assured that the hollow needle and particularly the cylindrical portion 102 do not function as an antenna and that the cylindrical portion 102 of the hollow needle 100 is not heated. Thereby, undesired side effects of the MR-imaging due to heating the cylindrical portion 102 up to burns of the patient are avoided. Preferably the hollow needle 100 and particularly the cylindrical portion 102 are made of plastics, carbon, carbon fiber and/or ceramics.

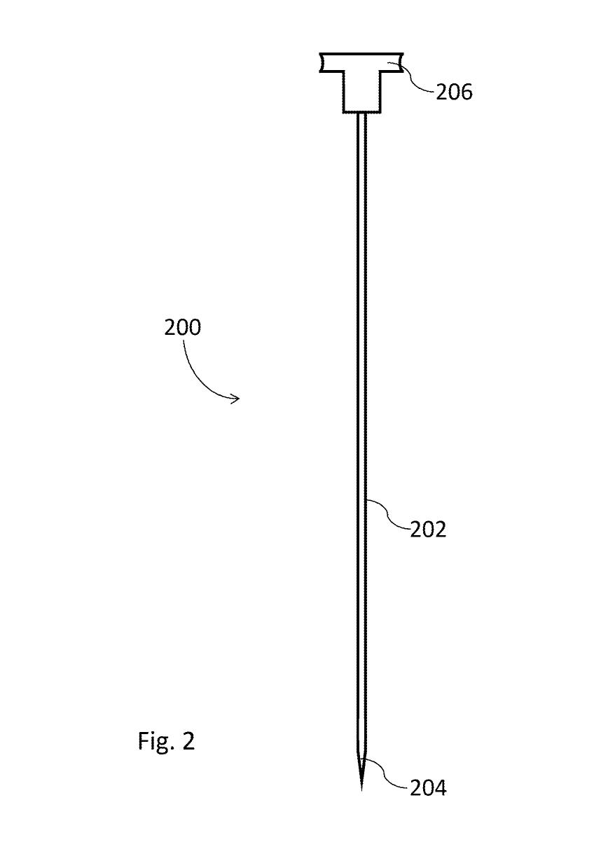

FIG. 2 shows a first mandrin used for biopsy and for introducing the hollow needle 100 in the body of a patient. The first mandrin 200 comprises a cylindrical portion 202, wherein on the distal end a sharp tip 204 and on its proximal end 206 a grip is located. The mandrin comprises for example in the field of prostate biopsy a length of approximately 16 cm to approximately 20 cm or more. The cylindrical portion 202 of the first mandrin has for example in the field of prostate biopsy a diameter of approximately 1.5 cm to approximately 2 cm. It is to be understood that a first mandrin for biopsy of other organs may have different dimensions. The cylindrical portion 202 and the tip 204 may be made of titanium or coated by titanium. Such mandrins are certified for biopsy and physicians have experience in use of such mandrins. Accordingly, the actual punction may be executed as used by physicians. The physician may control introducing the hollow needle 100 and the first mandrin 200 by the proximal grip 106 of the hollow needle 100 and of the proximal grip 206 of the first mandrin.

At the cylindrical portion 102 of the hollow needle 100 marks having a lower subdivision and marks 110 having a larger subdivision are arranged. A plurality of marks 108 having the lower subdivision coincide with a mark 110 having a larger subdivision. The marks 108 having a smaller subdivision may be spaced apart from the next mark 108 having the lower subdivision by 1 cm. A mark 110 having the larger subdivision may be spaced apart cm from the next mark having the larger subdivision 110. By means of the marks 108 having the lower subdivisions and the marks 110 having the larger subdivisions the physician can verify how deep the hollow needle 100 and the cylindrical portion 102, respectively of the hollow needle 100 have been introduced into the body.

The physician introduces the hollow needle 100 with inserted first mandrin 200, wherein the tip 204 of the first mandrin 200 projects from the distal opening 104 of the hollow needle 100, for a predetermined distance into the patient. Thereafter, the physician may verify using MR-imaging, whether the hollow needle 100 has been introduced in the correct direction. Thereby, imaging by a magnet resonance tomograph is carried out.

Since the first mandrin 200 comprises titanium or a titanium coating artifacts may arise and/or the first mandrin 200 may be heated, for example heated such that a burn of a patient occurs, if a magnet resonance tomograph having a magnetic field strengths of 3 Tesla or more is used.

Before imaging the first mandrin 200 having the sharp tip 204 is removed from the patient and the hollow needle 100 and an imaging mandrin 300 is introduced into the hollow needle. The imaging mandrin comprises a cylindrical portion 302, a grip 306 located at the proximal end of the cylindrical portion, and a distal end 304 located at the distal end of the cylindrical portion 302. In the interior of the cylindrical portion 302 a cavity 312 may be located which may also be formed cylindrical and extending from the blunt end 304 and the distal end 304, respectively in the direction of the grip 306. The cylindrical portion 302 may be made of plastics. The cavity 312 may be filled by a liquid, for example fish oil, water and/or a contrast agent. The cavity 312 extends as close as possible to the distal end 304, such that during imaging may be evaluated more accurately, where the hollow needle 100 is located in the body of a patient. Liquids, such as water, fish oil and/or contrast agent can be imaged by MR-imaging appropriately, whereas plastics, ceramics, carbon and/or carbon fibers are generally not appropriately imaged by such imaging. By removing the first mandrin 200 from the hollow needle and by introducing the imaging mandrin 300 into the hollow needle before imaging it can be assured that the patient is not impaired by a heated mandrin 200 and that the position of the hollow needle 100 may be located appropriately by virtue of the imaging mandrin 300 introduced therein.

Once the physician has verified the position of the hollow needle 100 in the body of the patient the imaging mandrin 300 may be removed from the hollow needle 100. Thereafter, the first mandrin 200 is again introduced by the grip 206 into the hollow needle and the hollow needle is moved in to the direction of the lesion as soon as the tip 204 projects from the hollow needle 100 at the distal end thereof.

The above described imaging may be repeated as many times as possible until the distal end 304 of the imaging mandrin has reached the lesion. Before each further imaging the first mandrin 200 is removed from the hollow needle 100 and the imaging mandrin 300 is introduced into the hollow needle. It is to be understood that after the imaging the imaging mandrin 300 is removed from the hollow needle 100 and the first mandrin 200 is introduced again into the hollow needle.

Concluding, the application of the present invention in medical diagnostic is described explanatory. The application of the present invention is particularly indicated, if as imaging modality a magnet resonance tomograph having a magnetic field strength of 3 Tesla or more, such as 7 Tesla, is used for the static magnetic field. The present invention may also be used in combination with other imaging modalities, for example with a ultrasonic modality or a computer tomograph modality.

During application of the present invention in combination with a magnet resonance tomograph the patient is lying on the table of the magnet resonance tomograph. Initially the table and the patient are moved into the static magnetic field of the magnet resonance tomograph, which is termed by chargon “to move into the tube”. Thereafter, RF-signals and gradient signals are applied and a MR-imaging is executed. By virtue of such imaging the physician may find an appropriate incision location for puncture.

After this first imaging the patient and the patient table are moved out of the static magnetic field and the magnet resonance tomograph, respectively and the physician applies a local anesthesia at the region to be punctured. Further, the physician can apply an incision in the skin of the patient such that the hollow needle 100 and the mandrin may be introduced into the patient more easily. The physician introduces the hollow needle 100 and the mandrin 200 into the body of the patient, wherein the sharp tip 204 of the mandrin projects from the hollow needle.

After a predetermined distance or a distance set by the physician along which the hollow needle 100 and the first mandrin 200 have been introduced into the body of the patient, the physician can execute a further imaging to ensure that the hollow needle 100 and the mandrin have been moved in the appropriate direction. Before executing the imaging, such as before the patient is moved into the static magnetic field of the magnet resonance tomograph and “into the tube” the physician may remove the first mandrin 200 having the sharp tip 204 from the hollow needle 100 and may introduce the imaging mandrin 300 with the fluid filled cavity 312 into the hollow needle. It is to be understood that the physician may remove the first mandrin from the hollow needle 100 and introduce the imaging mandrin 300 into the hollow needle 100 outside the static magnetic field of the magnet resonance tomograph. As soon as the imaging mandrin 300 is inserted into the hollow needle 100 and as soon as the patient is located within the static magnetic field of the magnet resonance tomograph and “within the tube” of the magnet resonance tomograph, a further imaging may be executed by the magnet resonance tomograph. The physician may judge based on the imaging, whether the hollow needle is located at the appropriate location within the body. The physician may remove the imaging mandrin outside the magnet resonance tomograph from the hollow needle 100 and introduce the first mandrin 200 having the sharp tip 204 again into the patient. If necessary, the physician may carry out corrections concerning the position of the hollow needle 100 or move the hollow needle 100 and the mandrin 200 into the direction of the lesion.

It is to be understood that the movement of the hollow needle 100 and of the first mandrin 200 in direction of the lesion and the consecutive imaging may carried out in an arbitrary number until the hollow needle is located at the correct position within the body of the patient. During this procedure the patient on the table of the magnet resonance tomograph may be moved into and out of the uniform static magnetic field of the magnet resonance tomograph, wherein introducing of the hollow needle 100 and of the first mandrin 200 are conducted outside the static uniform magnetic field and imaging is conducted within the static uniform magnetic field.

As soon as the hollow needle 100 is located at the position desired by the physician both the first mandrin 200 and the imaging mandrin 300 are removed from the hollow needle and the actual biopsy needle for conducting tissue removal may be introduced into the hollow needle 100.

The present invention has the advantage that an MR-guided biopsy is provided in which no artifacts in the region of the biopsy needle occur. Further, heating of the biopsy needle and impairment of the comfort of the patient or even a burn are avoided.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A biopsy needle system, comprising:
a hollow needle having a cylindrical portion adapted to be introduced into a patient, wherein the cylindrical portion comprises a first material; and
a first mandrin comprising a sharp tip and that may be introduced into the hollow needle such that the tip and an opposing end of the first mandrin project from the opposing end of the hollow needle, if the hollow needle is located in the patient, wherein the sharp tip is adapted to pierce tissue;
wherein an imaging mandrin comprising a second needle and that may be inserted into the hollow needle if the hollow needle is located in a patient, wherein the second material is a material that may be imaged appropriately during MR-imaging.

2. The biopsy needle system of claim 1, wherein the first material is a non-ferromagnetic material and/or a non-metallic material.

3. The biopsy needle system of claim 1, wherein the cylindrical portion of the hollow needle is formed of at least one of the materials selected from a list of materials consisting of: plastics; carbon; carbon fiber; ceramics and combinations thereof.

4. The biopsy needle system of claim 1, wherein the cylindrical portion of the imaging mandrin comprises plastics, wherein the cylindrical portion is adapted to be introduced into the hollow needle.

5. The biopsy needle system of claim 1, wherein the imaging mandrin comprises a cavity in which a liquid and/or a gel are located.

6. The biopsy needle system of claim 5, wherein the liquid and/or the gel comprises at least one liquid selected from a list consisting of: oil; fish oil; water; contrast agent; and combinations thereof.

7. The biopsy needle system of claim 1, wherein the imaging mandrin does not comprise a sharp tip.

8. A hollow needle for a biopsy needle system, comprising a cylindrical portion adapted to be introduced into a patient, wherein the cylindrical portion is formed of a non-ferromagnetic and/or nonmetallic material.

9. The hollow needle of claim 8, wherein the cylindrical portion of the hollow needle is formed of at least one of the materials selected from a list of materials consisting of: plastics; carbon; carbon fiber; ceramics and combinations thereof.

10. The hollow needle of claim 8, further comprising an imaging mandrin configured to be introduced into the hollow needle, wherein the imaging mandrin comprises a material that may be appropriately imaged by MR-imaging.

11. The hollow needle of claim 10, wherein a cylindrical portion of the imaging mandrin comprises plastics, wherein the cylindrical portion is adapted to be introduced into the hollow needle.

12. The hollow needle of claim 10, wherein the imaging mandrin comprises a cavity in which a liquid and/or a gel is located.

13. The hollow needle of claim 12, wherein the liquid and/or the gel comprises at least one liquid selected from a list consisting of: oil; fish oil; water; contrast agent; and combinations thereof.
